(19) United States
(12) Patent Application Publication
(10) Pub. No.: US 2008/0114067 A1
(43) Pub. Date: May 15, 2008

(54) Composition for Recovery From or Prevention of Central Nervous System Fatigue

(76) Inventor: Takanobu Yamamoto, Nara-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/883,173

(22) PCT Filed: Jan. 31, 2005
(86) PCT No.: PCT/JP05/01357

§ 371 Date: Jul. 27, 2007

(51) Int. Cl. A61K 31/198

(52) U.S. Cl. 514/561; 514/562; 514/563; 514/567

(57) ABSTRACT

A composition for recovery from or prevention of central nervous system fatigue (brain fatigue), comprising as active ingredients at least any amino acid selected from the group consisting of tyrosine, methionine, phenylalanine and glutamine, especially methionine and phenylalanine together with a branched chain amino acid of L-valine, L-leucine or L-isoleucine. This composition is provided as an injection agent or fluid infusion, or as, through addition of an appropriate vehicle such as starch or lactose, a solid dosable form (pharmaceutical application) such as tablets, granules or powder, or as any of various drinking water forms (food application) such as those known as health drink.

### TECHNICAL FIELD

The present invention relates to a composition for use in recovery from or prevention of fatigue in central nervous system, so-called brain fatigue.

### BACKGROUND ART

The present inventors have found that tryptophan is one of the causative substances which induce brain fatigue, and that administration of a branched-chain amino acid (BCAA) is effective for prevention of or recovery from brain fatigue, and filed a patent application for a preventive agent or a recovering agent (including a food) for brain fatigue, of which active ingredient is BCAA (see Patent Reference 1). In addition, the present inventors have found that concomitant use of an inhibitor in a blood-brain barrier L-system transporter, BCH (2-aminobicyclo[2,2,1]heptane-2-carboxylic acid) in addition to BCAA drastically suppresses brain fatigue, and filed a patent application for a concomitant drug using both agents as well (Patent Reference 1).

However, although it is suggested that BCH may be used as an anticancer drug, it has poor performance, and is currently difficult to be brought into practice.

On the other hand, results of a study about various amino acids as an agent to suppress uptake of L-tryptophan in a human placental cell have been reported (see Non-patent Reference 1). According to this study, uptake of tryptophan could be suppressed by L-phenylalanine, L-methionine, L-cysteine, L-leucine and L-valine. However, this report was about a placental brush border tissue.

In addition, although Non-patent Reference 2 indicates results of administration of BCAA together with glutamine and arginine to rats to carry out a tread mill running practice, other amino acids were not examined. Therefore, it would never be presumed whether or not the amino acids which suppress uptake of tryptophan as pointed out in the Non-patent Reference 1 actually contribute to suppression of brain fatigue.

Thus, the present inventor has proceeded a study about substances which are conventionally widely used in the field of food, as an alternative to BCH, and found that the above-mentioned amino acids, which are essential amino acids in human body same as BCAA, contribute to suppression of brain fatigue same as BCH, to complete the present invention.

### Patent Reference 1: International Publication WO 02/34257

### Non-patent Reference 1: Yoshiki Kudo and C. A. R. Boyd “Journal of Physiology” 2001, 531, 2, pp. 405-416

Non-patent Reference 2: Miro Smriga, Makiko Kameishi et al., “Preference for a Solution of Branched Chain Amino Acids plus Glutamine and Arginine Correlates with Free Running Activity in Rats: Involvement of Serotonergic Dependent Process of Lateral Hypothalamus” Nutritional Neuroscience International Journal on Nutrition, Diet and Nervous system, 2001.11.19, p 2002

### DISCLOSURE OF THE INVENTION

### Problems to be Solved by the Invention

Specifically, the present invention is to provide a highly safe and also highly effective composition that can be utilized as a food or a medicament officially approved to claim improving or suppressing fatigue in central nervous system (brain fatigue).

### Means for Solving the Problems

The composition for recovery from or prevention of fatigue in central nervous system of the present invention comprises any one or more kinds of amino acids selected from the group consisting of tyrosine, methionine, phenylalanine and glutamine together with a branched-chain amino acid as active ingredients, characterized in that the composition especially comprises BCAA together with tyrosine, and also glutamine in addition thereto, as active ingredients, or that the composition comprises BCAA together with methionine as well as phenylalanine, and tyrosine in addition thereto, as active ingredients. In other words, the present invention is targeted to taking concomitantly any one or more kinds of amino acids selected from the group consisting of tyrosine, methionine, phenylalanine and glutamine together with a branched-chain amino acid, among them, especially BCAA and tyrosine, BCAA and tyrosine and glutamine, or BCAA and methionine as well as phenylalanine, and tyrosine in addition to these three, for the purpose of recovery from or prevention of fatigue in central nervous system.

### EFFECT OF THE INVENTION

According to the present invention, improvement or prevention of brain fatigue which does not accompany physical fatigue and which is generated following, for example, computer works or works in the space environment which would become more active, can be carried out. Especially, since the composition of the present invention can be composed only of amino acids, it can be regarded that the safety of the composition is established, and the composition has high practical applicability.

### BEST MODE FOR CARRYING OUT THE INVENTION

The composition for recovery from or prevention of fatigue of the present invention comprises a branched-chain amino acid (BCAA), and any one or more kinds of amino acids selected from the group consisting of tyrosine, methionine, phenylalanine and glutamine as active ingredients. In the present invention, fatigue in central nervous system is same as what described in the above-mentioned Patent Reference 1, which is generated in a state not accompanying physical fatigue, such as computer works or readings, different from so-called feeling of fatigue generated following physical (muscular) fatigue. The prevention of fatigue of the present invention refers to a case which is applied mainly to a human prior to possible fatigue in central nervous system, and the recovery from fatigue refers to a case which is applied mainly to a human after generation of fatigue in central nervous system. In other words, the composition for recovery from or prevention of fatigue in central nervous system of the present invention can be applied regardless of presence or absence of fatigue, and expected to have novel applicability to recovery from or prevention of fatigue in central nervous system (brain fatigue), as a so-called food for specified health uses.

As a branched-chain amino acid (BCAA), an essential amino acid in a human body having a branched chain in a carbon chain such as L-valine, L-leucine or L-isoleucine. In addition, these amino acids can be used in the form of a physiologically acceptable salt, for example, a hydrochloride, or also in the form of various hydrates thereof. These branched-chain amino acids can be used alone individually, or used as a mixture. In addition, when used as a mixture, the mixing ratio thereof is not limited specifically.

An amino acid concomitantly used with BCAA includes L-tyrosine, L-methionine, L-phenylalanine and L-glutamine. While at least one kind of these amino acids is used, the amino acid is necessarily used concomitantly with BCAA. When only one kind of amino acid other than BCAA is concomitantly used, tyrosine is effective. In addition, the amino acid concomitantly used with BCAA may be a combination of two kinds of amino acids or a combination of three or more kinds of amino acids. Although the combination of two or more kinds is arbitrary, especially, a combination of L-tyrosine and L-glutamine, a combination of L-phenylalanine and L-methionine, and furthermore, a combination of L-phenylalanine, L-methionine and L-tyrosine are effective. Quantitative ratio of each amino acid in case of a combination can be appropriately determined.

Although compounding ratio of BCAA and an amino acid may also appropriately determined, the ratio is set to be BCAA-based, and the composition may have mass ratio of about 0.01 to 2, preferably about 0.1 to 1, regarding BCAA as 1. In addition, when plural kinds of other amino acids are used, the total amount in the above-mentioned range is sufficient. Of course, the compounding amount of other amino acids may be more than BCAA.

The form of the composition of the present invention to be taken or administered is not limited specifically, as long as the form is applicable to a human. The composition can be provided, for example, as an injectable agent or a transfusion which can be directly administered to vasculature or lymphoid system, and as a solid form which can be taken (for example, medical application) such as a tablet, a granulate or a powder, prepared by adding an appropriate excipient such as starch or lactose, and furthermore, as various drinkable forms (for example, food application) such as a so-called nutritional drink or the like. In addition, the composition may also be provided as a form for a so-called food in a form of a biscuit, a candy or the like.

Needless to say, to the composition for recovery from or prevention of fatigue of the present invention, various compounds conventionally used mainly for recovery from physical fatigue, such as sugars such as glucose or sucrose; B-complex vitamins such as vitamin B₁, B₂ or B₆; vitamin C; a metal ion such as a sodium ion or a potassium ion; or the like can be added, in addition to a branched-chain amino acid or other necessary amino acid. However, since tryptophan is a causative substance of brain fatigue and has a negative effect on the combined effect, a composition which does not comprise tryptophan is preferable.

The present invention will be explained further in detail hereinbelow with reference to examples described below. Needless to say, the present invention is not limited to the following examples.

### EXAMPLE 1

First, in accordance with the method described in the Patent Reference 1, a fatigue test wherein a female analbuminemia rat (Japan SLC, Inc.), which is genetically deficient of serum albumin, was forced to run on a treadmill was carried out. In other words, prior to the fatigue test, an analbuminemia rat grown at the room temperature of 22° C. under a light-dark cycle of 7:00 to 19:00 (light on) was forced to train (20 m/min, 7% tilt) for 30 minutes in a constant period between 13:00 and 15:00, four times per week for four weeks, to habituate to running on a treadmill. After completion of the training, all analbuminemia rats were forced to have exhaustive exercise on a treadmill under same conditions, and the running time up to exhaustion was measured. The exhaustion state was defined as the point of time at which the rat failed to follow the speed of the treadmill, or the point of time at which the rat refused to run.

The rats, which were divided into five groups (five rats per group) were respectively treated with each of physiological saline, BCAA, a concomitant drug of BCAA, phenylalanine and methionine (abbreviated as “BPM”), and a concomitant drug of BCAA, phenylalanine, methionine and tryptophan (abbreviated as “BPMT”). The physiological saline, phenylalanine, methionine, tryptophan and BCAA were intraperitoneally administered one hour before beginning of the exercise, in a dosage of 5 ml/kg, 125 mg/kg, 75 mg/kg, 85 mg/kg and 250 mg/kg, respectively. Here, BCAA and other each amino acid were dissolved in a physiological saline before use. In addition, a mixture of L-valine, L-leucine and L-isoleucine (weight ratio 5:3:2) was used as the BCAA. Incidentally, these dosages were determined expecting that an effect would be exhibited with the dosages, and actual dosage for a human can be appropriately varied depending on various factors. The results are shown in Table 1. In addition, as each amino acid, those for a food additive (manufactured by NICHIRI KAGAKU company) were used (same applies to Example 2).

TABLE 1
Running time up to exhaustion in rat treated with physiological
saline, BCAA, BPM and BPMT

| | Time up to exhaustion | |
|---|---|---|
| | (min) | Range of Time (min) |
| Saline (n = 5) | 248.6 ± 17.87 | 220-319 |
| BCAA (n = 5) | 359.8 ± 33.2*** | 240->420 (n = 1) |
| BPM (n = 5) | >420#** | >420 (n = 5) |
| BPMT (n = 5) | 347.2 ± 34.0* | 240->420 (n = 2) |

The table represents mean ± SEM.
#P = .0003: Significant difference was observed as compared with BCAA group (p value <0.005).
They were conducted with a Bonferroni/Dunn test in multigroup comparison.
*P = 0.0144,
**P = 0.0003,
***P = 0.00;
Significant difference was observed for physiological saline group in each case.

### They were conducted with a Fischer's PLSD test in multigroup comparison.

As can be found from these results, when phenylalanine and methionine were added (BPM), running time of the rat was drastically prolonged as compared with that in the case of administration of BCAA alone, and the effect was remarkable. In other words, although the rats could run only for four hours in the case of administration of a physiological saline and became able to run for about six hours in the case of administration of BCAA alone, the rats of PBM-administered group kept running on the treadmill tirelessly for seven hours or more. On the other hand, while it can be understood that the group to which tryptophan was added (BPMT) exhibited higher effect as compared with that of a physiological saline, the effect thereof lowered as compared with the BPM group so as to be the same extent as in the case of BCAA alone, and it was confirmed that tryptophan has an adverse effect.

### EXAMPLE 2

Next, the effect of the present invention was confirmed by having human individuals take the composition.

Four each of healthy male and female subjects of 20 to 21 years old were instructed to take a test drink. Thereafter, a test using multiple mood scale and a test of Profile of Mood States (Japanese edition of POMS) were conducted. The subjects were advised to refrain from eating or drinking, giving some serious thought, doing excessive exercise, or smoking, for 30 minutes after drinking the test drink, and they kept waiting. Subsequently, Uchida-Kraepelin test was performed in steps of 15 minutes of the first half, 3 minutes of resting and 15 minutes of the second half, to charge brain fatigue (acute brain fatigue). Immediately after the steps, the subjects were instructed to write in the mood immediately after the test in multiple mood scale items (Table 2). Thereafter, the subjects were instructed to write in mood of the past one week (chronic feeling of brain fatigue) by POMS test. The test took less than 80 minutes in total, comprising 30 minutes for the resting after drinking the test drink, 33 minutes for the Uchida-Kraepelin test and 10 minutes for writing in multiple mood scale items and POMS. Here, prior to the test, following explanation of the object of the test, precautions of the method or the like, a written agreement on a voluntary basis was obtained.

### [Test Drink]

BCAA, tyrosine, methionine, phenylalanine and glutamine were added to 200 ml of a commercially available orange juice (“natchan orange” manufactured by Suntory Limited: containing juice of orange and lemon, fructose, a flavoring agent, an acidulant and vitamin C; Here, nutrient component information per 100 ml is energy: 36 kcal, protein: 0.2 g, fat: 0 g, carbohydrate: 8.7 g, sodium: 0 mg and sucrose: 1.0 g.), respectively in an amount shown in Table 2. As the BCAA in this case, 1 g each of three kinds of branched-chain amino acids were used.

TABLE 2

| | | Phenylalanine | | Methionine | Glutamine |
|---|---|---|---|---|---|
| | BCAA (B) | (P) | Tyrosine (Y) | (M) | (G) |
| Test Group 1 | — | — | | — | — |
| (Control Group) | | | | | |
| Test Group 2 | 3 g | — | — | — | — |
| (BCAA Group) | | | | | |
| Test Group 3 | 3 g | — | 1 g | — | — |
| (BY Group) | | | | | |
| Test Group 4 | 3 g | 1 g | — | 1 g | — |
| (BMP Group) | | | | | |
| Test Group 5 | 3 g | 1 g | 1 g | 1 g | — |
| (BMPY Group) | | | | | |
| Test Group 6 | 3 g | — | 1 g | — | 1 g |
| (BYG Group) | | | | | |

### [Taking Test]

The test was conducted by instructing the subjects to drink 200 ml per test of drink, and ten times of tests in total, consisting of two times each of tests for the test groups 1 to 4 shown in Table 2 and one time each for the test groups 5 and 6, were conducted (the order of the test was as follows: test group 1→test group 2→test group 3→test group 4→test group 5→test group 1→test group 2→test group 3→test group 4→test group 6).

### [Fatigue Test 1: Multiple Mood Scale Test]

In the multiple mood scale test, the subjects were instructed to score each item of the multiple mood scale items for counting results shown in Table 3 (Terasaki, Masaharu et al., The Japanese Journal of Psychology, 1992, Vol. 62, No. 6, 350-356) as “feel”: 3 points, “feel a little”: 2 points, “not feel so much”: 1 point, or “not feel at all”: 0 point. Subsequently, as boredom items considered to be connected with brain fatigue (acute fatigue: rapidly tired), the items of Nos. 3, 11, 26, 36, 42, 48, 54, 64, 67 and 80 were selected, to calculate a score of the individual subject. In the multiple mood scale test, answer is made for evaluation at the time of answer, so that the answer to the selected items of the test evaluates acute fatigue. Here, upon conducting the actual test, the items in Table 3 was sorted in ascending order from No. 1 to No. 87.

TABLE 3
Multiple Mood Scale Items for Counting Results

| | Depression, | | | | | | | |
|---|---|---|---|---|---|---|---|---|
| No. | Anxiety | Score | No. | Concentration | Score | No. | Friendliness | Score |
| No 71 | Disturbed | | No | Cautious | | No | Dear | |
| | | | 53 | | | 77 | | |
| No 19 | Feeling | | No | Polite | | No | Lovely | |
| | Timid | | 58 | | | 51 | | |
| No 70 | Anxious | | No | Respectful | | No | Missing | |
| | | | 27 | | | 47 | | |
| No 17 | Worrying | | No | Thoughtful | | No | Fine | |
| | | | 21 | | | 60 | | |
| No 13 | Not | | No | Wary | | No | Favorite | |
| | Confident | | 76 | | | 85 | | |
| No 37 | Agonized | | No | Striving | | No | Dainty | |
| | | | 65 | | | 68 | | |
| No 2 | Pessimistic | | No 9 | Careful | | No | Longing | |
| | | | | | | 46 | | |
| No 79 | Depressed | | No | Serious | | No | Enrapt | |
| | | | 39 | | | 59 | | |
| No 44 | Moping | | No | Keen | | No | Pretty | |
| | | | 25 | | | 32 | | |
| No 87 | Melancholy | | No | Tense | | No | Merciful | |
| | | | 66 | | | 45 | | |
| No. | Boredom | Score | No. | Hostility | Score | No. | Startle | Score |
| No 48 | Bored | | No | Hostile | | No | Astonished | |
| | | | 75 | | | 29 | | |
| No 64 | Displeased | | No | Aggressive | | No 5 | Startled | |
| | | | 43 | | | | | |
| No 42 | Absurd | | No | Hateful | | No | Surprised | |
| | | | 82 | | | 38 | | |
| No 54 | Tired | | No | Defiant | | No 6 | Amazed | |
| | | | 15 | | | | | |
| No 80 | Dull | | No | Rancorous | | No | Frightened | |
| | | | 57 | | | 16 | | |
| No 36 | Listless | | No | Huffy | | No | Tingled | |
| | | | 61 | | | 12 | | |
| No 26 | Feckless | | No | Raging | | No | Dithered | |
| | | | 69 | | | 56 | | |
| No 67 | Vacant | | No | Angry | | No | Excited | |
| | | | 20 | | | 84 | | |
| No 11 | Careless | | No | Offended | | No | Panicked | |
| | | | 18 | | | 40 | | |
| No 3 | Indifferent | | No | Irritated | | No | Stunned | |
| | | | 30 | | | 22 | | |

| No. | Well-Being | Score | No. | Liveliness | Score |
|---|---|---|---|---|---|
| No 62 | Easygoing | | No 7 | Lively | |
| No 52 | Unhurried | | No | Vigorous | |
| | | | 10 | | |
| No 34 | Tranquil | | No 8 | Energetic | |
| No 81 | Gentle | | No 1 | Vivid | |
| No 24 | Carefree | | No | Smooth | |
| | | | 41 | | |
| No 74 | Softened | | No | Pleasant | |
| | | | 83 | | |
| No 49 | Placid | | No | Comfortable | |
| | | | 35 | | |
| No 14 | Patient | | No | Good-Humored | |
| | | | 50 | | |
| No 4 | Relaxed | | No | Merry | |
| | | | 55 | | |
| No 31 | Unstrained | | No | Refreshed | |
| | | | 86 | | |

### [Fatigue Test 2: POMS Test]

In POMS, the subjects were instructed to score 65 items described in Japanese Edition of POMS (Yokoyama, Kazuhito et al., Japanese edition of POMS No. 851, 2002, KANEKO SHOBO) in five grades from “Not at all (score 0)” to “Extremely (score 4).” Accordingly, a score about F items indicating reduction in motivation and vitality (“tired,” “reluctant to doing something,” “racked,” “hacked,” “listless,” “tired of something” and “used-up”) was obtained according to the evaluation described in the POMS. In POMS, different from the multiple mood scale test, answer is given for evaluation in a period from one week before the test to the time point of the answer, so that the answer to the selected items evaluates chronic fatigue.

### [Test Result]

Test results of the multiple mood scale test are shown in Table 4 and 5, and test results of the POMS test are shown in Table 6. Here, efficacy ratio is shown by means of the number of subjects whose score was decreased as compared with that of test group 1 (control group).

TABLE 4
Table of Score of Multiple Mood Scale (Partially Modified)
in Fatigue Test (Acute Fatigue)
First Time

| | | | | | | | | | Efficacy |
|---|---|---|---|---|---|---|---|---|---|
| Subject | A | B | C | D | E | F | G | H | Ratio |
| Test Group 1 (Control | 10 | 9 | 10 | 13 | 9 | 11 | 7 | 13 | — |
| Group) | | | | | | | | | |
| Test Group 2 (BCAA | 13 | 9 | 8 | 10 | 2 | 9 | 2 | 7 | 6/8 (75%) |
| Group) | | | | | | | | | |
| Test Group 3 (BY | 7 | 2 | 14 | 7 | 15 | 14 | 7 | 4 | 4/8 (50%) |
| Group) | | | | | | | | | |
| Test Group 4 (BMP | 8 | 10 | 11 | 16 | 13 | 20 | 1 | 2 | 3/8 (38%) |
| Group) | | | | | | | | | |
| Test Group 5 (BMPY | 8 | 11 | 12 | 7 | 11 | 3 | 21 | 3 | 4/8 (50%) |
| Group) | | | | | | | | | |

TABLE 5
Table of Score of Multiple Mood Scale (Partially Modified)
in Fatigue Test (Acute Fatigue)
Second Time

| | | | | | | | | | Efficacy |
|---|---|---|---|---|---|---|---|---|---|
| Subject | A | B | C | D | E | F | G | H | Ratio |
| Test Group 1 (Control | 10 | 14 | 15 | 10 | 18 | 5 | 6 | 5 | — |
| Group) | | | | | | | | | |
| Test Group 2 (BCAA | 7 | 12 | 7 | 11 | 14 | 14 | 7 | 4 | 5/8 (63%) |
| Group) | | | | | | | | | |
| Test Group 3 (BY | 9 | 14 | 11 | 15 | 19 | 8 | 0 | 4 | 4/8 (50%) |
| Group) | | | | | | | | | |
| Test Group 4 (BMP | 11 | 2 | 12 | 17 | 11 | 7 | 13 | 4 | 4/8 (50%) |
| Group) | | | | | | | | | |
| Test Group 5 (BYG | 8 | 4 | 13 | 15 | 15 | 9 | 7 | 4 | 5/8 (63%) |
| Group) | | | | | | | | | |

TABLE 6
Table of Score of Items Indicating Reduction in Motivation and Vitality (by POMS Test) in Fatigue Test (Chronic Fatigue)

| | | | | | | | | | | | | | | | | | Efficacy |
|---|---|---|---|---|---|---|---|---|---|---|---|---|---|---|---|---|---|
| Subject | A-1 | B-1 | C-1 | D-1 | E-1 | F-1 | G-1 | H-1 | A-2 | B-2 | C-2 | D-2 | E-2 | F-2 | G-2 | H-2 | Ratio |
| Test Group | 7 | 10 | 12 | 17 | 21 | 24 | 1 | 5 | 3 | 2 | 9 | 18 | 12 | 14 | 4 | 5 | — |
| 1 (Control | | | | | | | | | | | | | | | | | |
| Group) | | | | | | | | | | | | | | | | | |
| Test Group | 11 | 13 | 21 | 21 | 14 | 18 | 10 | 6 | 8 | 8 | 12 | 17 | 7 | 14 | 3 | 2 | 6/16 (38%) |
| 2 (BCAA | | | | | | | | | | | | | | | | | |
| Group) | | | | | | | | | | | | | | | | | |
| Test Group | 6 | 10 | 21 | 17 | 14 | 15 | 23 | 6 | 6 | 12 | 9 | 21 | 13 | 14 | 1 | 8 | 4/16 (25%) |
| 3 (BY | | | | | | | | | | | | | | | | | |
| Group) | | | | | | | | | | | | | | | | | |
| Test Group | 9 | 1 | 19 | 11 | 13 | 14 | 14 | 2 | 9 | 0 | 11 | 22 | 9 | 8 | 26 | 5 | 8/16 (50%) |
| 4 (BMP | | | | | | | | | | | | | | | | | |
| Group) | | | | | | | | | | | | | | | | | |
| Test Group | 11 | 13 | 16 | 17 | 12 | 19 | 17 | 4 | — | — | — | — | — | — | — | — | 3/8 (38%) |
| 5 (BMPY | | | | | | | | | | | | | | | | | |
| Group) | | | | | | | | | | | | | | | | | |
| Test Group | — | — | — | — | — | — | — | — | 5 | 6 | 10 | 21 | 7 | 17 | 16 | 7 | 1/8 (13%) |
| 6 (BYG | | | | | | | | | | | | | | | | | |
| Group) | | | | | | | | | | | | | | | | | |

* Subject A-1 means the test of the first time, and A-2 means the test of the second time. Same applies to subject B and later.

It can be found from the results of the animal experiments that concomitant use of an amino acid such as phenylalanine or methionine can drastically prolong the running time on the treadmill as compared with that in the case of use of BCAA alone. In addition, in experiments in human, the efficacy ratio was 50% or more, for example, for acute brain fatigue, when tyrosine was added to BCAA (BY group), when tyrosine and glutamine were added to BCAA (BYG group), phenylalanine and methionine were added to BCAA (BMP group), and when tyrosine was additionally added to the BMP group (BMPY group) (see Tables 4 and 5). Moreover, the efficacy ratio was 50% or more for chronic brain fatigue, when phenylalanine and methionine were added to BCAA (BMP group) (see Table 6). It can be understood from these animal experiments and experiments in human that an additive effect is high especially when phenylalanine and methionine are added to BCAA.

### INDUSTRIAL APPLICABILITY

The composition for recovery from or prevention of fatigue of the present invention exhibits effective suppression or improvement for brain fatigue showing symptoms different from those of physical fatigue, and provides a so-called functional food, a medicament or the like capable of advocating new efficacy and effect.

1. A composition for recovery from or prevention of fatigue in central nervous system, comprising any one or more kinds of amino acids selected from the group consisting of tyrosine, methionine, phenylalanine and glutamine, and a branched-chain amino acid, as active ingredients.

2. A composition for recovery from or prevention of fatigue in central nervous system, wherein active ingredients are tyrosine and a branched-chain amino acid.

3. A composition for recovery from or prevention of fatigue in central nervous system, wherein active ingredients are tyrosine, glutamine and a branched-chain amino acid.

4. A composition for recovery from or prevention of fatigue in central nervous system, wherein active ingredients are phenylalanine, methionine and a branched-chain amino acid.

5. The composition for recovery from or prevention of fatigue in central nervous system according to claim 4, further comprising tyrosine as an active ingredient.

6. Dose regimen of taking any one or more kinds of amino acids selected from the group consisting of tyrosine, methionine, phenylalanine and glutamine, and a branched-chain amino acids, for recovery from or prevention of fatigue in central nervous system.

7. Dose regimen of taking tyrosine and one or more branched-chain amino acids, for recovery from or prevention of fatigue in central nervous system.

8. Dose regimen of taking tyrosine, glutamine and a branched-chain amino acid, for recovery from or prevention of fatigue in central nervous system.

9. Dose regimen of taking phenylalanine, methionine and one or more branched-chain amino acids, for recovery from or prevention of fatigue in central nervous system.

10. The dose regimen according to claim 9, wherein tylosin is further concomitantly taken.
